(19) United States
(12) Patent Application Publication
(10) Pub. No.: US 2004/0002697 A1
(43) Pub. Date: Jan. 1, 2004

(54) Biconic ablation with controlled spherical aberration

(76) Inventors: Gerhard Youssefi, Landshut (DE); Friedrich Moritz, Muenchen (DE)

Correspondence Address: Bausch & Lomb Incorporated, One Bausch & Lomb Place, Rochester, NY, 14604-2701, US

(21) Appl. No.: 10/460,801

(22) Filed: Jun. 12, 2003

Related U.S. Application Data
(60) Provisional application No. 60/392,243, filed on Jun. 27, 2002.

(51) Int. Cl. A61B 18/20; A61F 9/007; A61B 19/00

(52) U.S. Cl. 606/5; 606/10; 128/898

(57) ABSTRACT

A laser vision correction ablation algorithm relies upon the central radius of curvature and a biconic shape factor of a pre-operative and a post-operative anterior corneal surface. The post-operative shape factor is selected to provide a spherical aberration value that is optimized for a particular patient or for a particular patient population group. The algorithm is embodied as a readable, executable instruction in a device readable medium. The algorithm further sets forth a method for laser vision correction.

### BACKGROUND OF THE INVENTION

1. Field of the Invention

The concept of the invention is generally directed to the field of laser vision correction and, more particularly, to apparatus, algorithms, and methods that provide control of spherical aberration associated with a laser vision correction procedure.

2. Description of Related Art

The field of laser vision correction currently offers several types of procedures for correcting or improving refractive defects by laser photoablation of the corneal surface. These procedures include PRK, LASIK, and LASEK, which are typically used to correct myopic and hyperopic defects with or without astigmatism, and in some cases provide customized treatments to address at least some of the higher order aberrations of the eye.

A well known technique for delivering a conventional myopic LASIK treatment is the Planoscan® ablation algorithm delivered by the Technolas 217A® laser system (Bausch & Lomb Incorporated, Rochester, N.Y.). In this system, selected scanning patterns of a 2 mm diameter laser beam are used to ablate the corneal surface. The interested reader is referred to U.S. Pat. Nos. 6,090,100 and 5,683,379, which are herein incorporated by reference in their entirety to the full extent allowed, by applicable laws and rules.

For some time, laser manufacturers have been developing their ablation algorithms based upon the so called Munnerlyn approach and the well known equation bearing his name for determining ablation depth as a function of optical zone size. According to Munnerlyn et al., the cornea is modeled as two refracting surfaces with an intervening bulk material of index, n. For myopic (i.e., near-sighted) correction, the goal was to increase the anterior radius of curvature, or flatten the central anterior cornea, as illustrated in FIG. 1. A simple geometric formula described the “shape subtraction” paradigm, which based the final corneal shape on the amount of tissue subtracted by the laser from the initial corneal shape. With the use of the Munnerlyn formula, the volume to be ablated, A, (described as the nominal ablation) is calculated as follows:

$$A = \sqrt{{R}_{Pre}^{2} - {x}^{2}} - \sqrt{{R}_{Pre}^{2} - 0.25 ( {OZ}^{2} )} - \sqrt{{R}_{Post}^{2} - {x}^{2}} + \sqrt{{R}_{Post}^{2} - 0.25 ( {OZ}^{2} )}$$

Where

A: Ablation in μm

x: Distance from center during treatment

Rₚᵣₑ: Pre-op radius of curvature of the cornea

Rₚₒₛₜ: Post-op radius of curvature of the cornea

OZ: Optical zone diameter (i.e., the desired size of the corrected region on the cornea).

The Munnerlyn equation, for example, serves as a starting point for many ablation algorithms. For a myopic ablation, the pre-operative cornea is modeled as a sphere of greater curvature than the desired post-operative cornea, which is also modeled as a sphere. In order to simplify computation of the nominal ablation, the software may assume that the pre-operative radius of curvature is the same for all eyes. (The mean value of the population is 43.4 D or effectively 7.8 mm). The apex of the desired post-operative cornea is displaced from the pre-operative cornea until the desired optical zone is reached, thus determining the maximum ablation depth. Parameters useful for the computation of the nominal ablation include the size of the individual laser spot, its energy profile (i.e., the change of intensity or energy of a laser spot as a function of the radius), as well as the amount of tissue ablated by one pulse (i.e., the rate of ablation). The Planoscan algorithm, for example, uses a laser spot having a beam diameter of 2 mm at the target, as well as a so-called “flat-top” profile. This means that the intensity or energy in this laser spot is substantially uniform across about 90% or more of the beam profile. At completion of these computation steps a treatment plan in the form of a pulse file is created that is intended to result in a desired refractive myopic correction.

Laser vision correction treatments, however, typically induce residual spherical aberration. Residual spherical aberration may result from an OZ that is smaller than the patient's dilated pupil, causing glare and halo effects in low-light conditions, or from a spherical post-operative corneal surface. This issue has been recognized in the prior art, along with observations and measurements indicating that the pre-operative corneal surface is not spheroid, but rather is a prolate ellipsoid. Myopic correction involving flattening of the central corneal region typically results in an oblate ellipsoid that again exhibits spherical aberration.

In view of the foregoing, the inventors have recognized a need for overcoming the limitations and concerns discussed above in providing improved vision through laser vision correction.

### SUMMARY

An embodiment of the invention is directed to an algorithm for laser vision correction. The algorithm fundamentally determines a resultant corneal profile expressed in terms of a pulse file (i.e., a calculated sequence of individual laser beam pulse locations over an ablation area of the cornea). The file may subsequently be processed by a suitable laser vision correction laser system in order to achieve a refractively effective change in the shape of the cornea. The general components of the algorithm include determining pre-operative surface parameters of the cornea such as a pre-operative central radius of curvature, R, and a pre-operative shape factor, Q; determining the desired post-operative refractive correction, D (diopters); determining a desired post-operative central radius of curvature, R′, from the desired refractive correction, D, and the pre-operative central radius of curvature, R; and determining a desired post-operative, biconic shape factor, Q′(x,y), that provides a targeted post-operative spherical aberration value. In an aspect of this embodiment, the targeted post-operative spherical aberration value can be optimized for a particular patient or for a particular patient population using, for example, statistical methods. In another aspect, the vision correction ablation is carried out with only 2 mm laser beam diameter pulses having either a Gaussian or a truncated-Gaussian (soft-spot as that term will be used herein) energy profile, or, with only 2 mm and 1 mm laser beam diameter pulses having either a Gaussian or a truncated-Gaussian energy profile (these pulse diameters being merely exemplary). In another aspect, a residual corneal thickness determination can enable/disable the vision correction treatment.

Another embodiment of the invention is directed to a device readable medium having stored therein an algorithm as outlined above, or alternatively, an executable instruction for directing a laser vision correction system to deliver a vision correction treatment implementing the algorithm outlined above.

Another embodiment of the invention is directed to a method for laser vision correction comprising carrying out the steps of the algorithm outlined above.

Objects and advantages of the embodiments of the invention will be further appreciated by those skilled in the art in view of the detailed description and drawings that follow, and by the appended claims which solely define the invention.

### BRIEF DESCRIPTION OF THE DRAWINGS

The accompanying drawings, which are incorporated in and constitute a part of this specification, illustrate embodiments of the present invention and together with the description serve to explain the principals of the invention. In the drawings,

FIG. 1 is an illustration of a myopic correction of a cornea known in the prior art;

FIG. 2 shows a flow chart describing the components of the algorithm according to an embodiment of the invention;

FIG. 3 shows a flow chart describing additional components of the algorithm according to an aspect of the invention;

FIG. 4 shows a flow chart describing additional components of the algorithm according to another aspect of the invention;

FIG. 5 is a block diagram of a laser vision correction system including a device readable medium according to an embodiment of the invention;

FIG. 6 is an illustration of a laser beam profile associated with an embodiment of the invention;

FIG. 7 is an enlarged photocopy of a laser beam profile shaping aperture associated with an embodiment of the invention;

FIG. 8 schematically illustrates an idealized uniform ablation of a target;

FIG. 9 schematically illustrates an actual target ablation in contrast to the idealized ablation illustrated in FIG. 8; and

FIG. 10 is a chart illustrating different aspects of the pre-and post-operative shape factors of a cornea according to an embodiment of the invention.

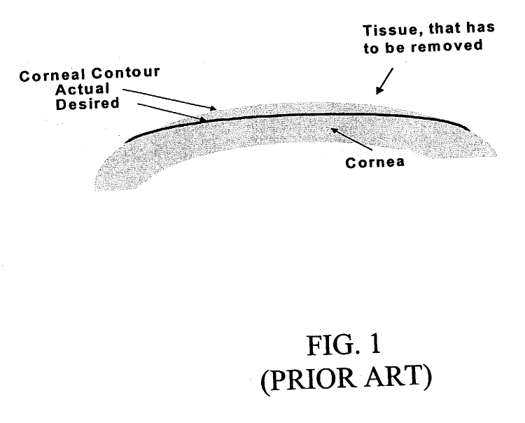

### DETAILED DESCRIPTION OF A PREFERRED EMBODIMENT

Embodiments of the invention are directed to an algorithm for laser vision correction; a computer or device readable medium having stored therein the algorithm, or an executable instruction for directing a laser vision correcting platform to execute the algorithm; and to a method for laser vision correction, with supporting apparatus. The various embodiments are now described with reference to the Figures, where like reference numerals are used to refer to like elements throughout.

As may be used in this application, the term “computer component” refers to a computer-related entity, hardware, firmware, software, a combination thereof, or software in execution. For example, a computer component can be, but is not limited to being, a process running on a processor, a processor, an object, an executable, a thread of execution, a program and a computer. By way of illustration, both an application running on a server and the server can be computer components. One or more computer components can reside within a process and/or thread of execution and a computer component can be localized on one computer and/or distributed between two or more computers. The term “software,” as may be used herein, includes but is not limited to, one or more computer readable and/or executable instructions that cause a computer or other electronic device to perform functions, actions and/or behave in a desired manner. The instructions may be embodied in various forms like routines, algorithms, modules, methods, threads, and/or programs. Software may also be implemented in a variety of executable and/or loadable forms including, but not limited to, a stand-alone program, a function call (local and/or remote), a servelet, an applet, instructions stored in a memory, part of an operating system or browser, and the like. It is to be appreciated that the computer readable and/or executable instructions can be located in one computer component and/or distributed between two or more communicating, co-operating, and/or parallel processing computer components and thus can be loaded and/or executed in serial, parallel, and other manners.

For the purpose of ease of understanding, the embodied inventive methodologies are illustrated and described as a series of blocks, which are not necessarily limited to their illustrated order or concurrence. Moreover, less than all of the illustrated blocks in a figure may be sufficient to implement a particular methodology. Additionally, the methodologies may be implemented as computer executable instructions and/or operations stored on computer readable media including, but not limited to, an application specific integrated circuit (ASIC), a compact disc (CD), a digital versitle disk (DVD), a random access memory (RAM), a read only memory (ROM), a programmable read only memory (PROM), an electronically erasable programmable read only memory (EEPROM), a disk, a carrier wave, and a memory stick.

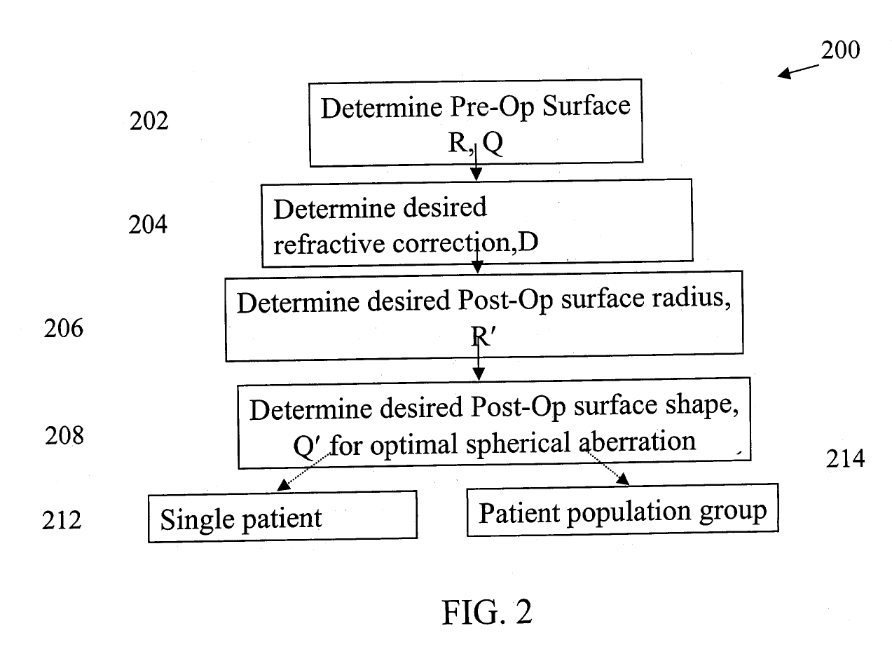

FIG. 2 illustrates in flow chart format the basic components of an algorithm 200 according to an embodiment of the invention. At box 202, the pre-operative, anterior corneal central radius of curvature, R, and the pre-operative, anterior corneal shape, Q, are determined. Commercially available topography devices or ophthalmometers can provide direct readings or allow the experimental determination of both parameters. Unlike the Munnerlyn paradigm referred to above, the values R and Q will generally define a conical surface defined by

$$Z = \frac{{p}^{2}}{R + √ [ {R}^{2} - ( 1 + Q ) {p}^{2} ]}$$

Where

Z is the sag of the conical surface,

p²=x²+y²,

R=central radius of curvature, and

−1≦Q≦1(Q≠0), where the surface can be a prolate or oblate ellipsoid, a

parabola, or a hyperbola.

In an aspect of this embodiment, the conic constants Q (and Q′) define biconic surfaces; i.e., Q (and Q′) and the central radius of curvature, R (and R′), are functions of x,y, and may be different in the x and y directions. A biconic surface allows specification of Rₓ, R_{y}, Qₓ, Q_{y} (as well as their respective post-operative values) directly. As those skilled in the art will understand, the sag, Z, of a biconic can be expressed as

$$Z = \frac{{c}_{x} {x}^{2} + {c}_{y} {y}^{2}}{1 + √ [ 1 - ( 1 + {Q}_{x} ) {c}_{x}^{2} {x}^{2} - ( 1 + {Q}_{y} ) {c}_{y}^{2} {y}^{2} ]}$$

$$where {c}_{z}^{biconic} = \frac{- {s}_{x} {c}_{x} {x}^{2} - {s}_{y} {c}_{y} {y}^{2}}{{c}_{x} {x}^{2} + {c}_{y} {y}^{2}}$$

$$and {R}_{z}^{biconic} = \frac{+ {c}_{x} {x}^{2} + {c}_{y} {y}^{2}}{- {s}_{x} {c}_{x} {x}^{2} - {s}_{y} {c}_{y} {y}^{2}}$$

$$and \underline{{s}_{x}} = - ( 1 + {Q}_{x} ) , \underline{{s}_{y}} = - ( 1 + {Q}_{y} )$$

$$so that - {z}^{2} {c}_{z}^{biconic} = - 2 z + ( {c}_{x} {x}^{2} + {c}_{y} {y}^{2} ) . \\ Substituting z = {z}^{′} + {R}_{z}^{biconic}$$

$$then , {c}_{x} {x}^{2} + {c}_{y} {y}^{2} + {c}_{z}^{biconic} {z}^{′ 2} = 1 / {c}_{z}^{biconic}$$

$$and since {c}_{x} = 1 / {R}_{x} , {c}_{y} = 1 / {R}_{y} , \\ then {x}^{2} / {R}_{x} + {y}^{2} / {R}_{y} + {z}^{′2} / {R}_{z}^{biconic} = {R}_{z}^{biconic} . \\ Employing the definitions {c}_{z} = \frac{- {s}_{x} {c}_{x} - {s}_{y} {c}_{y}}{2}$$

$$and Δ = \frac{- {s}_{x} {c}_{x} + {s}_{y} {c}_{y}}{2}$$

$$gives ( in series expanded form ) {c}_{z}^{biconic} = \frac{- {s}_{x} {c}_{x}^{2} {x}^{2} - {s}_{y} {c}_{y}^{2} {y}^{2}}{{c}_{x} {x}^{2} + {c}_{y} {y}^{2}} \\ = \frac{{c}_{x} ( {c}_{z} + Δ ) {x}^{2} + {c}_{y} ( {c}_{z} - Δ ) {y}^{2}}{{c}_{x} {x}^{2} + {c}_{y} {y}^{2}} \\ = {c}_{z} - \frac{Δ ( {c}_{x} {x}^{2} - {c}_{y} {y}^{2} )}{{c}_{x} {x}^{2} + {c}_{y} {y}^{2}} .$$

The interested reader is further directed to MacRae et al., Customized Corneal Ablation, p.102, SLACK (2001), which is incorporated herein by reference to the extent allowed by applicable laws and rules. One skilled in the art will appreciate that the biconic model can be used to define different Q′ values for half-meridians (e.g., meridian 1 at 10°, meridian 2 at 100°, from a Q1 and Q2 for meridian 1, and a Q3 and Q4 for meridian 2). Next, the desired refractive correction, D (in diopters) is determined at box 204. This can be accomplished by subjective manifest refraction or objective refraction with the use of commercially available phoropters, refractometers, software fitted topographers and/or wavefront analyzers, and other devices. Once D is known, the desired post-operative anterior central radius of curvature, R′, is determined, as shown at 206. This is preferably accomplished using the formula R′=(n−1)/(Dₚᵣₑ₋ₒₚ−D′), where n is the corneal index of refraction. Along with R′, a post-operative anterior corneal shape, Q′, can be selected, at 208, to optimize the amount of post-operative residual spherical aberration. In one aspect, this optimization can be selected at 212 for the individual patient, depending upon age, occupation, comfort, and other factors that will help to provide the patient with the highest level of patient satisfaction. In another aspect 214, Q′ can be selected to optimize the residual spherical aberration for a large patient population group based upon statistical analysis, for example. In an alternative aspect pertaining to the biconic description of orthogonal meridians, R′ and Q′ values could be defined for different areas of the cornea (e.g., set 1 for a central area, set 2 for a peripheral ring 1, set 3 for peripheral ring 2, and so on). It will be appreciated by a person skilled in the art that a growing understanding of the role of spherical aberration in vision quality will drive empirical and analytical optimization.

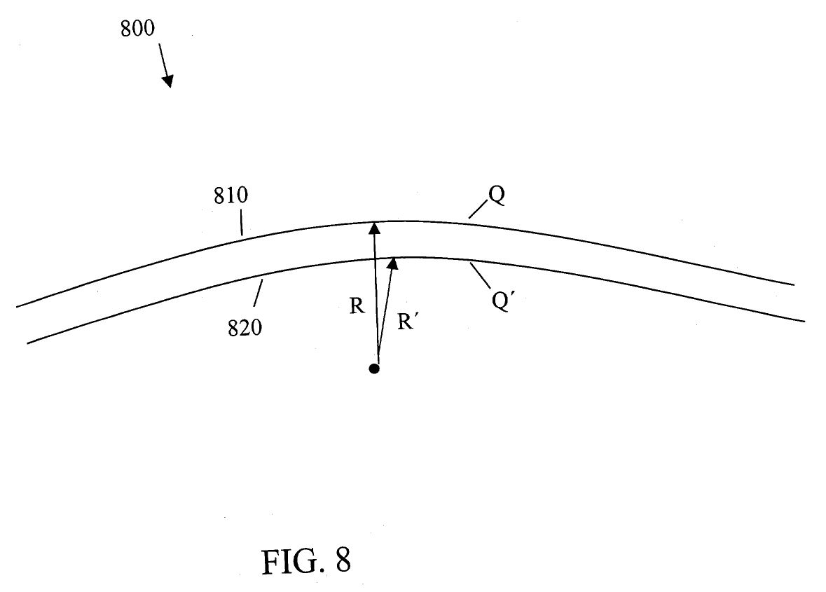

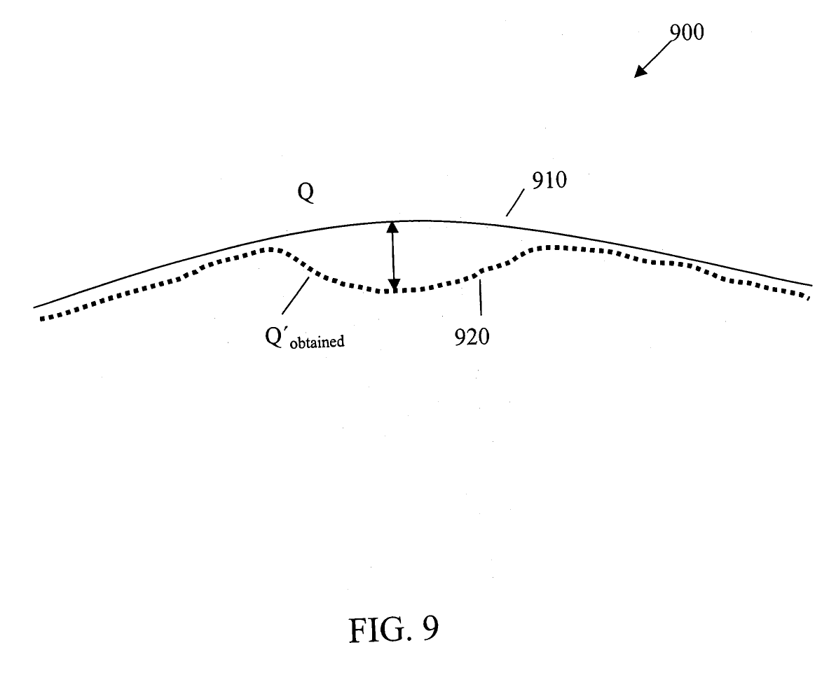

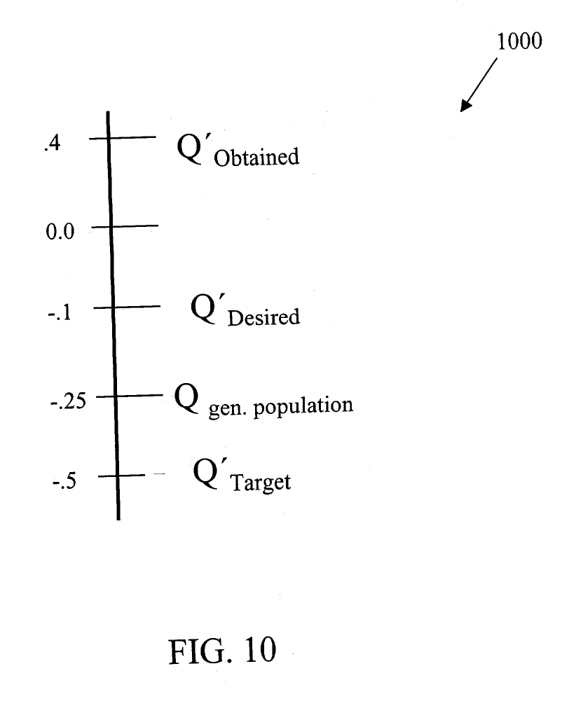

One may wish to provide a scaling factor for the asphericity correction, which could be based on corneal thickness, corneal architecture (e.g., pachymetry profile), the shape of the cornea, age, sex, type and amount of treatment (e.g., myopia, hyperopia), and final corneal curvature. For example, assume a patient has a typical pre-operative corneal shape factor, Q=−0.25, and requires a refractive correction of −5D with a desired post-operative Q′=−0.1 and R′=R (i.e., same pre- and post-operative central radii of curvature). Due to one or more factors relating to degree of correction, biodynamic effects, age effects, and/or other physiologic factors, the desired post-operative shape factor, Q′, will not necessarily be directly obtained after ablation without adjustment or scaling. FIG. 8 provides an exaggerated illustration of a “plastic” cornea 800 showing the pre-operative corneal profile 810 and the post-operative corneal profile 820 for R=R′. This situation is never realized. Rather, the obtained post-operative shape might appear as illustrated (not to scale) in FIG. 9 where 910 is the pre-operative corneal profile and 920 represents the actually obtained post-operative corneal profile Q′_{obtained}=0.4 without any adjustment or scaling. In this example, the surgeon would choose a target value Q′_{target}=−0.5 to get a Q′_{desired}=−0.1. This is further illustrated in FIG. 10, which shows values on a linear scale 1000 for Qₚᵣₑ₋ₒₚ, Q′_{desired}, Q′_{obtained}, and Q′_{target}. This indicates that the proper selection of a Q′_{target} value will most likely be determined empirically based upon clinical experience and surgeon adjusted nomograms.

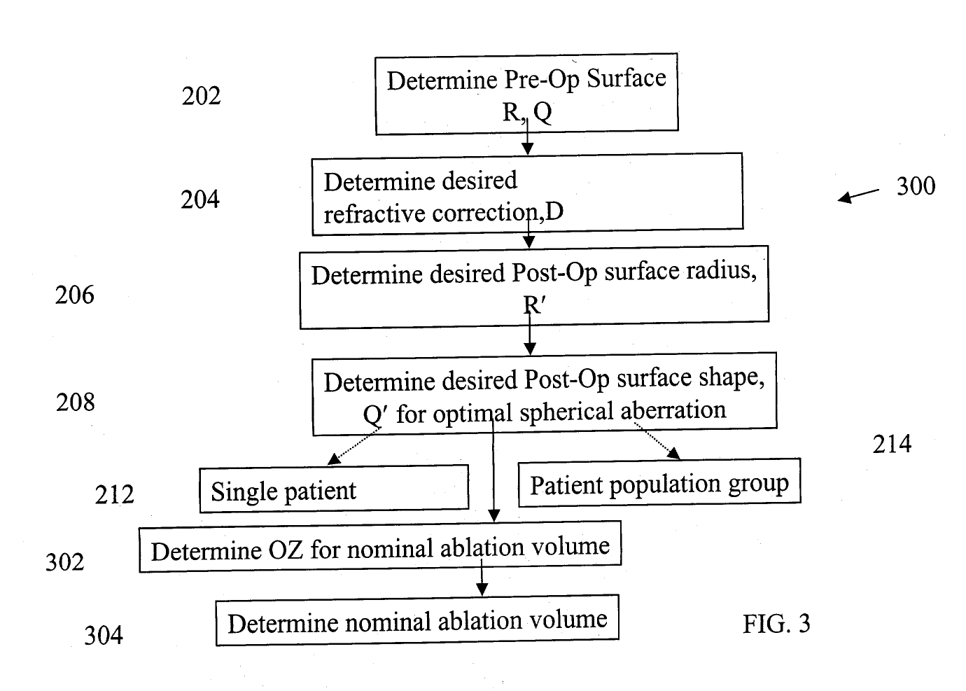

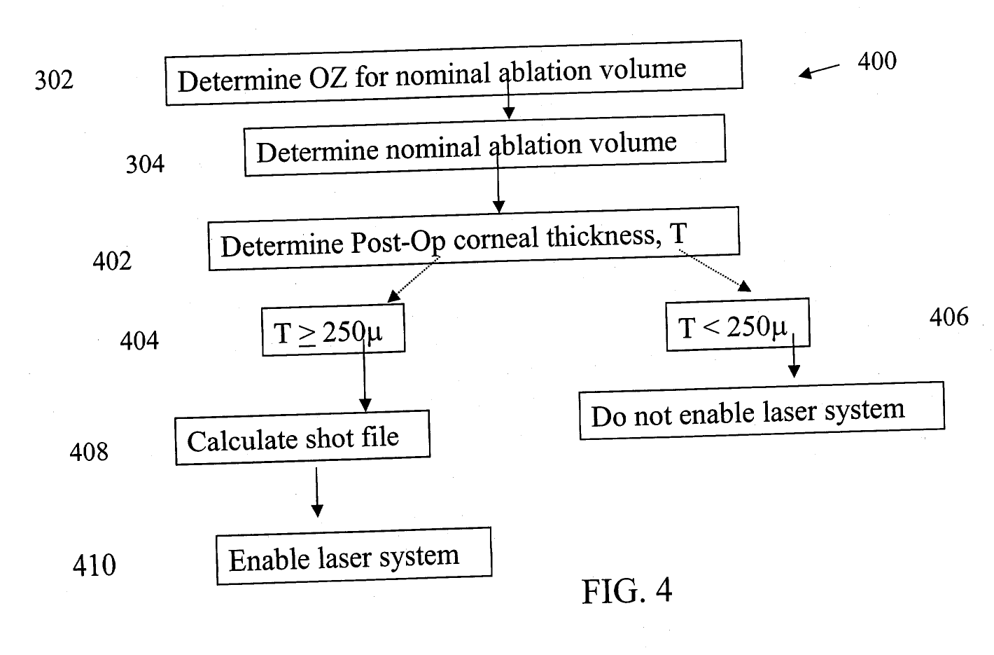

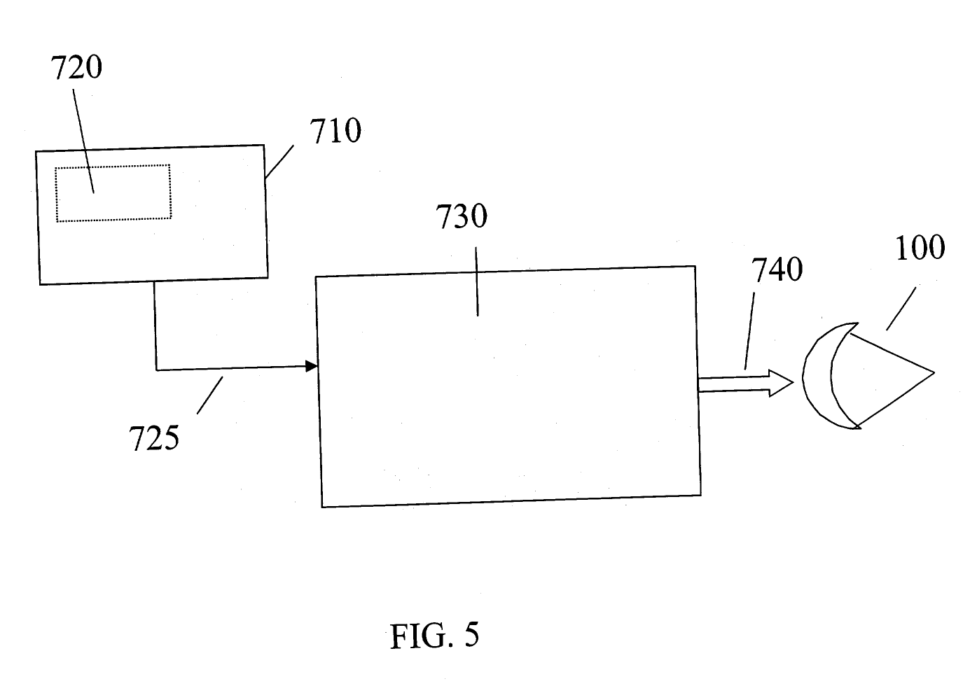

As illustrated by box 302 in FIG. 3, the optical zone for the nominal ablation is determined. This follows along the procedure used in the Munnerlyn-type approach described above. The calculated post-operative surface is shifted (ablation volume increased) until the desired OZ is reached. The nominal ablation volume simply results from the difference between the pre-operative and post-operative surfaces. A software routine, herein referred to as Proscan™ software, similar to Planoscan software described above, calculates a laser pulse file at 408 in FIG. 4, to fill the nominal ablation volume. It may be desired, as shown at box 402, to determine a prospective post-operative corneal thickness, T, prior to calculating the pulse file. Under a reasonable standard of care, corneal ablation is contraindicated when the residual stromal thickness will be less than 200 μm, and more typically when T<250 μm (box 406). However, if T>about 250 μm, then the laser pulse file at 408 can be calculated and the laser system controllably enabled at box 410.

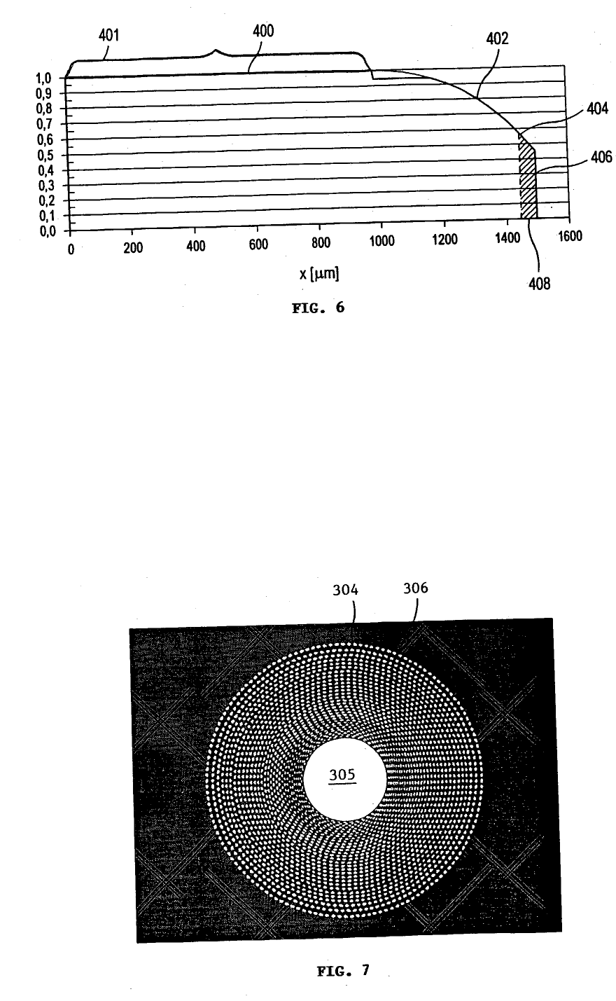

Parameters controlling the laser pulse file calculation 408 include the laser beam size and shape on the target surface, the laser beam energy profile, the amount of tissue ablated per pulse, laser pulse repetition rate, scanning patterns, beam overlapping, and others. In an aspect of the embodiment, the target beam includes a combination of only 2 mm diameter and 1 mm diameter on-target beams having “soft-spot” energy profiles. This combination of beam sizes provides time-efficient ablation and the ability to more efficiently correct for higher frequency, higher order aberrations in addition to defocus and cylinder. The term “soft-spot” herein refers to a laser beam profile 400 as shown graphically in FIG. 6. In the figure, the profile is normalized and only one-half the profile 400 is illustrated, solely for simplicity of the drawing, it being understood that the full profile 400 would be as if mirrored about the ordinate axis of FIG. 6. As can be seen, a center portion 401 of the aperture profile 400 is flat or substantially flat, whereas an edge 402 of the profile 400 is continuous with the portion 401 and is rounded. The portion 401 is symmetric about the radius of the profile and extends across about 60-80% of the profile 400 in one aspect, and across about 65-70% of the profile 400 in another aspect. At a certain point, such as an intensity threshold point 404 at which the eye tissue ablation intensity threshold is no longer reached, the profile 400 quickly drops off or diminishes as a substantially square, vertical, or truncated edge 406. The ablation threshold and any variations in it are known in the art. The amount of energy falling below the threshold for ablation is intended to be about 5% or less of the total energy encompassed by the profile 400. The profile 400 is non-Gaussian, between square and Gaussian-shaped, known as a truncated Gaussian. The soft-spot beam profile can be formed by passing the laser output through what is referred to as a “soft-spot” aperture 306, as shown in FIG. 7. The soft-spot aperture 603 is defined herein as having a larger central, directly transmitting portion 605 surrounded by a multiplicity of microscopic subapertures 603 which diffractively transmit and shape the beam and produce a desired beam intensity profile 400, i.e., in the form of a truncated Gaussian. An aperture card (not shown) preferably has two soft-spot apertures of different overall diameters, preferably in the range of 1 mm to 3 mm. Upon proper alignment and positioning of the card in the laser beam path, two different beam spot sizes can alternately be projected onto the exposed cornea surface. The profile 400 in FIG. 6 has an ordinate dimension (diameter) of 3 mm since the overall beam diameter at the target surface is demagnified to 2 mm. The interested reader is referred to U.S. Pat. Nos. 6,090,100; 5,683,379; 5,827,264; 5,891,132, for detailed information about the soft-spot aperture and soft-spot profile, all of which are herein incorporated by reference in their entirety to the extent allowed by applicable laws and rules. The foregoing described beam sizes, shapes, and profiles are not intended to be limiting examples, but are merely illustrative beam parameters. Single beam sizes only, dual beam sizes only, or other beam size combinations can be used in the range of between about 0.5 mm to 7 mm.

Another embodiment according to the invention, shown with reference to FIG. 7, is directed to a device readable medium 710 for use with a laser vision correction system. In an aspect of the embodiment, the medium 710 is in the form of an enablement-type card having stored therein an executable instruction 720 for directing an ophthalmic laser platform 730 to deliver a nominal ablation 740 in an optical zone of the corneal surface. The particular architecture of the executable instruction 720 can take various forms. It may comprise software that is downloadable by the laser platform that instructs it to deliver the ablation. In this case, the instruction would include all, or at least a part of, the algorithm 200, 300, 400 according to the invention. Alternatively, the medium may contain a code that can match a pre-programmed routine resident in the laser platform whereupon matching the instruction code with the resident instruction will enable the laser platform to execute the ablation. This mode would facilitate a card medium 710 with a simple, low capacity data storage (e.g., 1000 bytes). More details of this aspect of a device readable medium are contained in co-owned and co-pending application entitled Ophthalmic Correction Apparatus and Method for Improving Vision, filed concurrently with the instant priority application.

In another embodiment according to the invention, a method for providing a laser vision correction comprises all aspects of the algorithm methodologies described in detail above, which are set forth here by reference.

Notwithstanding the embodiments specifically illustrated and described herein, it will be appreciated that various modifications and variations of the foregoing embodiments are possible in light of the description set forth above and the appended claims, without departing from the spirit and scope of the invention as a whole.

We claim:

1. A laser vision correction ablation algorithm, comprising:
determining a pre-operative surface of the cornea from information consisting of a pre-operative central radius of curvature, R, and a pre-operative shape factor, Q;
determining a desired refractive correction, D;
determining a desired post-operative surface having a central radius of curvature, R′, and a desired post-operative shape factor, Q′, wherein Q′ is a biconic shape factor.

2. The algorithm of claim 1, wherein Q′ is selected to effect a desired post-operative spherical aberration value.

3. The algorithm of claim 1, wherein R and Q are multiple R and Q values for respective multiple orthogonal meridians, and comprising determining respective R′ and Q′ values.

4. The algorithm of claim 1, wherein determining R′ and Q′ further comprises determining a plurality of R′ and/or Q′ values corresponding to different regions on the cornea.

5. The algorithm of claim 4, wherein the different regions include at least a central region and peripheral region.

6. The algorithm of claim 1, wherein determining Q′ comprises determining a scaled value of Q′ to account for at least one of a corneal thickness, a corneal architecture, a corneal shape, a patient's age, a patient's gender, a type and amount of treatment, and a final corneal curvature.

7. The algorithm of claim 6, wherein determining a scaled value of Q′ comprises selecting a target value Q′_{T} different from the desired Q′.

8. The algorithm of claim 7, wherein Q′_{T} is an empirically determined value.

9. The algorithm of claim 2, wherein the desired post-operative spherical aberration value is an optimal value for a particular patient.

10. The algorithm of claim 2, wherein the desired post-operative spherical aberration value is an optimal value for a particular patient population group.

11. The algorithm of claim 1, further comprising determining an optical zone size for a nominal ablation volume of the cornea.

12. The algorithm of claim 11, comprising determining the nominal ablation volume by shifting the post-operative surface from the pre-operative surface until the optical zone size is reached.

13. The algorithm of claim 12, comprising calculating a laser pulse file for the nominal ablation volume.

14. The algorithm of claim 13, comprising using only single diameter laser beam pulses to calculate the pulse file.

15. The algorithm of claim 13, comprising using only two different diameter laser beam pulses to calculate the shot file.

16. The algorithm of claim 1, wherein the algorithm further comprises determining a post-operative, residual corneal thickness.

17. The algorithm of claim 1, wherein the algorithm further comprises determining whether the post-operative, residual stromal thickness will be equal to or greater than a predetermined value.

18. The algorithm of claim 17, wherein the predetermined value is nominally 250 microns.

19. The algorithm of claim 17, wherein the algorithm further comprises releasing a fire control lock in the laser vision correction system if the determination is positive.

20. A device readable medium for use with a laser vision correction system having stored therein a readable instruction for directing the laser vision correction system to execute an algorithm, said algorithm comprising:
determining a pre-operative surface of the cornea from information consisting of a pre-operative central radius of curvature, R, and a pre-operative shape factor, Q;
determining a desired refractive correction, D;
determining a desired post-operative surface having a central radius of curvature, R′, and a desired post-operative shape factor, Q′, wherein Q′ is a biconic shape factor.

21. The device readable medium of claim 20, wherein Q′ is selected to effect a desired post-operative spherical aberration value.

22. The device readable medium of claim 21, wherein the desired post-operative spherical aberration value is an optimal value for a particular patient.

23. The device readable medium of claim 21, wherein the desired post-operative spherical aberration value is an optimal value for a particular patient population group.

24. The device readable medium of claim 20, wherein the algorithm further comprises determining an optical zone size for a nominal ablation volume of the cornea.

25. The device readable medium of claim 24, wherein the algorithm further comprises determining the nominal ablation volume by shifting the post-operative surface from the pre-operative surface until the optical zone size is reached.

26. The device readable medium of claim 24, wherein the algorithm further comprises calculating a laser shot file to fill the nominal ablation volume.

27. The device readable medium of claim 20, wherein the algorithm further comprises determining a post-operative, residual stromal thickness.

28. The device readable medium of claim 27, wherein the algorithm further comprises determining whether the post-operative, residual stromal thickness will be equal to or greater than a predetermined value.

29. The device readable medium of claim 28, wherein the predetermined value is nominally 250 microns.

30. The device readable medium of claim 28, wherein the algorithm further comprises releasing a fire control lock in the laser vision correction system if the determination is positive.

31. The device readable medium of claim 20, wherein determining Q′ comprises determining a scaled value of Q′ to account for at least one of a corneal thickness, a corneal architecture, a corneal shape, a patient's age, a patient's gender, a type and amount of treatment, and a final corneal curvature.

32. The device readable medium of claim 31, wherein determining a scaled value of Q′ comprises selecting a target value Q′_{T} different from the desired Q′.

33. The device readable medium of claim 32, wherein Q′_{T} is an empirically determined value.

34. A method for providing a laser vision correction, comprising:
determining a pre-operative surface of the cornea from information consisting of a pre-operative central radius of curvature, R, and a pre-operative shape factor, Q;
determining a desired refractive correction, D;
determining a desired post-operative surface having a central radius of curvature, R′, and a desired post-operative shape factor, Q′, wherein Q′ is a biconic shape factor.

35. The method of claim 34, wherein Q′ is selected to effect a desired post-operative spherical aberration value.

36. The method of claim 34, wherein R and Q are multiple R and Q values for respective multiple orthogonal meridians, and comprising determining respective R′ and Q′ values.

37. The method of claim 34, wherein determining R′ and Q′ further comprises determining a plurality of R′ and/or Q′ values corresponding to different regions on the cornea.

38. The method of claim 37, wherein the different regions include at least a central region and peripheral region.

38. The method of claim 34, wherein determining Q′ comprises determining a scaled value of Q′ to account for at least one of a corneal thickness, a corneal architecture, a corneal shape, a patient's age, a patient's gender, a type and amount of treatment, and a final corneal curvature

39. The method of claim 35, wherein the desired post-operative spherical aberration value is an optimal value for a particular patient.

40. The method of claim 35, wherein the desired post-operative spherical aberration value is an optimal value for a particular patient population group.

41. The method of claim 34, further comprising determining an optical zone size for a nominal ablation volume of the cornea.

42. The method of claim 41, comprising determining the nominal ablation volume by shifting the post-operative surface from the pre-operative surface until the optical zone size is reached.

43. The method of claim 41, comprising calculating a laser pulse file for the nominal ablation volume.

44. The method of claim 38, wherein determining a scaled value of Q′ comprises selecting a target value Q′_{T} different from the desired Q′.

45. The method of claim 44, wherein Q′_{T} is an empirically determined value.

46. The method of claim 43, comprising using only a single diameter laser beam pulses to calculate the shot file.

47. The method of claim 43, comprising using only two diameter laser beam pulses to calculate the shot file.

48. The method of claim 34, further comprising determining a post-operative, residual stromal thickness.

49. The method of claim 48, further comprising determining whether the post-operative, residual stromal thickness will be equal to or greater than a predetermined value.

50. The method of claim 49, wherein the predetermined value is nominally 250 microns.

51. The method of claim 49, further comprising, releasing a fire control lock in the laser vision correction system if the determination is positive.
